(19) United States
(12) Patent Application Publication
(10) Pub. No.: US 2015/0250969 A1
(43) Pub. Date: Sep. 10, 2015

(54) AIRWAY OPENING APPARATUS AND METHOD

(71) Applicant: MONDO MEDICAL DEVICES PTY LTD, Coolum Beach, QL (AU)

(72) Inventors: Marcus Maria Xavier, Biggera Waters (AU); Warren John Tegg, Coolum Beach (AU); Karl Adrian Mills, Mountain Creek (AU)

(21) Appl. No.: 14/417,883

(22) PCT Filed: Jul. 15, 2013
(86) PCT No.: PCT/AU2013/000783

§ 371 Date: Jan. 28, 2015

(30) Foreign Application Priority Data
Aug. 3, 2012 (AU) 2012903352

(51) Int. Cl. A61M 16/04; A61M 16/08; A61B 19/08

(52) U.S. Cl. CPC A61M 16/0497; A61M 16/0434; A61B 19/08; A61M 16/0816

(57) ABSTRACT

An apparatus for use in providing an airway through a tracheal wall of a subject, the apparatus including, a dilator including a handle, wherein the handle allows a user to hold and manipulate at least the dilator, and wherein the dilator provides a channel extending from a proximal end to a distal end, and, a sleeve detachably mounted to the dilator, the sleeve being for receiving a tracheal tube having an airway running from a proximal end to a distal end, and in use the dilator can be used to insert the tracheal tube through the tracheal wall with the tracheal tube being detachable from the sleeve to allow the dilator to be removed so that the tracheal tube provides an airway through the tracheal wall.

### BACKGROUND OF THE INVENTION

The present invention relates to an apparatus and method for providing an airway through a tracheal wall of a subject, and in one particular example to an apparatus and method suitable for performing a percutaneous cricothyrotomy or percutaneous tracheostomy procedure.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Cricothyrotomy and tracheostomy procedures are surgical procedures to open an airway through a subject's cricothyroid membrane or trachea, respectively. Such procedures are used in situations when the subject's airway is obstructed or the subject is otherwise unable to breathe through their mouth or nasal passages, and are often required in emergency settings to prevent asphyxiation of the subject in those situations.

Early tracheostomy procedures were invasive and required relatively large incisions through the tracheal wall to provide a sufficiently sized opening to allow a tracheostomy tube to be inserted. Percutaneous techniques have since been developed, which require only a single small incision or needle puncture through the subject's skin and tracheal wall, and these techniques generally help to reduce trauma and post-operative complications to the subject.

Commonly used percutaneous techniques typically involve the initial use of a syringe with a needle to provide a hole through the tracheal wall. After the hole is provided, a guide wire is advanced into the trachea in order to guide the insertion of a pre-dilator. Following pre-dilation, a number of further separate pieces of equipment may be used including dilating equipment such as multiple tapered dilators utilised in sequence, for gradually dilating the hole as they are inserted.

The use of many separate pieces of medical equipment makes the procedures relatively complicated and time consuming. Furthermore, the procedures typically require a medical professional with considerable skill and dexterity to operate the different pieces of equipment when performing the procedure. This can be particularly problematic in the emergency settings where such procedures are often required, as timeliness and precision can mean the difference between life and death.

In CN-2887259 a disposable induction emergency tracheotomy device is three-sleeve structure, wherein an outer sleeve and an inner sleeve are covered mutually, a fixed belt at the upper end part of the outer sleeve is provided with a fixed belt hole and the upper end part of the inner sleeve is provided with a sponge body with small holes at one side. The diameter of the inner sleeve lower end reduces gradually to form a cone-shaped shape, one side at the tail end is provided with a blade which extends out of a groove, and in the groove is provided with a semi-circular blade. The guide wires drill through the center of the inner sleeve and are connected with a hollow puncture needle at the lower end part, while the puncture needle drills through the cone-shaped tail end port of the inner sleeve. the utility model is simple in structure, easy to operate and easy for mass production; in addition, the utility model will not cause the infection and the spread of the virus due to the single use, which can be widely used in the medical clinical treatments and field emergency treatments.

In U.S. Pat. No. 4,488,545 a catheter placement device for use in introducing high frequency jet ventilation gas to the trachea of a patient is provided which includes a catheter, and a catheter introducer having a needle within the catheter and includes an actuating member for moving the needle to an extended position in which the needle tip extends beyond the distal end of the catheter for percutaneously inserting the catheter and needle into the throat of a patient. A spring moves the needle tip to a retracted position within the catheter upon release of the actuating member. The introducer is removable from the catheter so that a source of high frequency ventilation gas can be connected to the catheter. The introducer has a piston which is movable in a bore which communicates with the needle lumen.

In WO-2007/006055 an intravenous catheter insertion device and method of use are described. The insertion device coordinates movement of an access needle, a coaxial intravenous catheter and a flexible safety guidewire. A vein is punctured with the access needle, then an actuation member on the insertion device is used to advance the safety guidewire into the vein. The safety guidewire allows the access needle and the intravenous catheter to be safely advanced into the vein. Then, the actuation member is activated to simultaneously withdraw the access needle and the safety guidewire, leaving only the intravenous catheter in the vein. The intravenous catheter is then disconnected from the insertion device and connected to a source of intravenous fluid, medication, etc.

U.S. Pat. No. 5,944,732 teaches a subcutaneous tunneling device, a dilator and a method of forming a subcutaneous tunnel. The device includes a trocar having a front end with a point configured for piercing skin, and a dilator which has a front end removably attached to the rear end of the trocar. The dilator is for dilating a section of a subcutaneous tunnel and includes a dilating section having a maximum diameter sized for dilating an interior surface of a subcutaneous tunnel for seating a catheter stabilizing cuff in the subcutaneous tunnel. The method of forming a subcutaneous tunnel includes the steps of inserting a trocar through a first location on a cutaneous surface and moving the trocar through subcutaneous tissue to form a subcutaneous tunnel. The trocar is at least partially removed and a front end of a dilator is attached to the trocar. A section of the tunnel is dilated by moving the dilator partially through the tunnel until the front end of the dilator reaches a first subcutaneous location in the tunnel. The dilator and trocar are at least partially withdrawn from the dilated tunnel section of the subcutaneous tunnel through the first end of the tunnel.

WO-99/38548 discloses a multiple lumen endotracheal tube having a main lumen (12), an inflatable cuff (28) concentrically formed around the main lumen (12) near its distal end (13), a cuff inflation lumen (24) in fluid communication with the cuff (28), a dedicated suction lumen (20) having a plurality of stacked suction eyelets (22) formed near the end of the suction lumen (20) directly above the cuff (28) and suction trigger (30) in fluid communication with the suction lumen (20) for aspirating secretions pooled in the trachea above the cuff (28). The suction and cuff inflation lumens (20, 28) comprise passages extending longitudinally through the wall of the main lumen (12) and tube extensions extending outside the main lumen. A lavage port (16) is connected in fluid communication to the suction lumen (20) for selectively and intermittently irrigating the trachea above the cuff (28) prior to aspiration of pooled secretions. The multiple suction eyelets (22) are strategically placed to prevent the cuff (28) from being drawn up against the suction eyelets (22) during aspiration and to provide access to the suction lumen (20) when one or more of the suction eyelets (22) are blocked.

WO-2006/125006 teaches a tracheostomy performed using an access device and a separate ventilation device. The access device is introduced through a surgical opening in the tracheal wall and has an anchor which is expanded in situ to hold the access device in place. The ventilation device is introduced through a passage in the access device and has an expandable cuff which is oriented above the access point through the tracheal wall. A concavity in the expandable cuff collects body secretions, and other materials from the oral and nasal cavities and/or gastro-intestinal reflux into the trachea, and the collected secretions are removed by aspiration through a lumen provided in the ventilation device. A one-way valve may be provided in the expandable cuff in order to permit exhalation through the larynx to assist in speech.

In US-2006/0124134 a tracheostomy system includes an outer multi-layered tube, which can be expanded or allowed to contract as necessary in order to receive various sizes of cannula tubes. A dilator is used to initially insert the outer tracheostomy tube into the tracheostoma. After the initial installation dilator is removed, various sizes of dilators having a cannula mounted about them can be inserted into the outer tracheostomy tube. The multilayered tube will then expand in response to insertion of the various sizes of dilator cannula assembly being placed. When the dilator is removed the cannula tube will remain in place to maintain the desired diameter tracheostomy tube. This provides a means in which the diameter of the tube can be changed without having to actually remove and reinsert a different tube.

U.S. Pat. No. 5,217,005 discloses a percutaneous device for performing tracheotomies or cricothyroidectomies having a hollow needle with a sharp distal end. The device includes a flexible dilator slidably positioned over the needle. The dilator includes a conical portion and an annular groove, the apex of which is positioned adjacent the distal end of the needle and the base thereby terminating at the annular groove. A flexible breathing tube is slidably positioned over the dilator for insertion into a trachea or larynx.

U.S. Pat. No. 7,036,510 teaches an apparatus and method for performing a percutaneous tracheostomy procedure utilizing dilatation means that do not require entry into the trachea by downward pressure, in order to minimize the risk of posterior tracheal trauma. In addition, the apparatus is structured to facilitate entry of the tracheostomy tube into the dilated entry site without permitting any significant shrinkage or reduction in diameter of the dilated opening.

WO-2005/094926 describes an apparatus for use in the percutaneous placement of a medical device, such as a tracheostomy tube. An elongated hollow tube (14) has an inflatable dilator balloon (12) mounted thereon. The balloon comprises a distal portion (16), an intermediate portion (17) and a proximal portion (18). The medical device is carried on the intermediate portion. At least a segment of the distal portion has an inflated outer diameter that is at least as large as the outer diameter of the medical device. The inflated outer diameter of the intermediate portion is sized relative to an internal diameter of the medica device to hold the medical device thereon. An inflation assembly (49) is provided to enable the balloon to be selectively inflated and deflated. A body opening is dilated with the inflated dilator balloon, and the medical device may be percutaneously placed across the dilated opening. Following placement of the device, the dilator balloon may be deflated and withdrawn from the device through a lumen of the medical device.

In CN-201299631, disclosed is a percutaneous intercricocentesis trocar, which comprises a flexible sleeve, a puncture needle, an interlocking wire with certain rigidity and a puncture needle operating part. One end of the interlocking wire is connected with the puncture needle operating part and the other end is linked with the puncture needle. Further the puncture needle and the interlocking wire are sleeved in the flexible sleeve. The utility model is simple in structure, safe and effective, simple and easy to learn, little in damage and low in cost. When the percutaneous intercricocentesis trocar is used in intercricocentesis ventilation emergency process, specialist physicians in anesthesiology department, otolaryngology department or pneumology department are not needed, ordinary medical workers after a short period of training are capable of executing puncture ventilation for patients at the very first time, thereby improving overall cardiopulmonary cerebral resuscitation prognosis.

CN-2067129 relates to a rapid trachea lancing device which is a medical first-aid apparatus. The rapid trachea lancing device is used for lancing the trachea within one minute in emergency rescue so as to establish artificial ventilation. The rapid trachea lancing device comprises an inner core and an outer sleeve pipe, wherein, the inner core comprises a handle, a movable clamp for fixing the inner core and the outer sleeve pipe, an inner conduit pipe (which forms the radian of physiological curvature of the trachea) and a miniature knife; the outer sleeve pipe comprises a pipe shaft, a pipe seat, an air bag, an air injecting pipe, a fixed handle, etc. The rapid trachea lancing device is matched with a special connector which can be connected with various artificial ventilation apparatuses at home and abroad, and the upper part is provided with a sputum sucking hole and a medicine injecting piston. The artificial ventilation does not need to be interrupted during medication inside the trachea, sputum suction, etc. The method of the combination of cutting and penetration is adopted by the apparatus, and skin and trachea cartilaginous rings are penetrated rapidly. The outer sleeve pipe and the inner core enter the trachea simultaneously. The artificial ventilation of any methods can be carried out by opening the movable clamp, taking out the inner core and injecting air into the air bag.

WO-2008/034872 teaches a device and a method for the percutaneous placement of a tracheostomy tube composed of a handle, an inflatable balloon having a reversed truncated cone shape, a tube to inflate the balloon and another to contain a wire guide, a plastic structure in the middle between the balloon and the handle made of laminar elements to strengthen the apparatus movements of the handle transmitted to the tube and the balloon. Following placement of the tracheal tube, the balloon is deflated and the apparatus or device withdrawn.

### SUMMARY OF THE PRESENT INVENTION

The present invention seeks to ameliorate one or more of the problems associated with the prior art.

In a first broad form the present invention seeks to provide an apparatus for use in providing an airway through a tracheal wall of a subject, the apparatus including:
a) a dilator including a handle, wherein the handle allows a user to hold and manipulate at least the dilator, and wherein the dilator provides a channel extending from a proximal end to a distal end; and
b) a sleeve detachably mounted to the dilator, the sleeve being for receiving a tracheal tube having an airway running from a proximal end to a distal end, and in use the dilator can be used to insert the tracheal tube through the tracheal wall with the tracheal tube being detachable from the sleeve to allow the dilator to be removed so that the tracheal tube provides an airway through the tracheal wall.

Typically the dilator includes a shoulder, wherein the shoulder defines a mounting for receiving the sleeve.

Typically the shoulder includes at least one of the following:
a) A substantially annular shape;
b) A graduated profile; and,
c) A substantially corrugated profile.

Typically the dilator further includes a body including a dilating portion for dilating the tracheal wall of the subject.

Typically the body includes an elongated shaft.

Typically the dilating portion is substantially frustoconical.

Typically the sleeve is mounted to the dilator using any one of:
a) an interference fit;
b) a friction fit; and,
c) a clip fit.

Typically the sleeve includes a tapered portion for dilating the tracheal wall of the subject.

Typically the tapered portion aligns with a dilating portion of the dilator, wherein the tapered portion and the dilating portion are for dilating the tracheal wall of the subject.

Typically the apparatus includes a detachment member, wherein the detachment member enables detachment of the sleeve and the tracheal tube.

Typically the sleeve includes a flange, wherein in use the detachment member is positioned between the tracheal tube and the flange, thereby urging apart the tracheal tube and the flange and detaching the sleeve and the tracheal tube.

Typically the detachment member includes a substantially graduated profile.

Typically the detachment member is substantially “U” shaped.

Typically the detachment member enables detachment of the sleeve and the dilator.

Typically the dilator includes a collar and wherein, in use, the detachment member is positioned between the sleeve and the collar, thereby urging apart the sleeve and the collar and detaching the sleeve and the dilator.

Typically the detachment member is any one of the following:
a) detached from the apparatus;
b) attached to any one of the sleeve and the dilator; and,
c) integrally formed with any one of the sleeve and the dilator.

Typically the apparatus includes a guide provided through the channel, thus allowing insertion of the apparatus through the tracheal wall to be guided by the guide.

Typically the guide includes a guide wire.

Typically an outer surface of the apparatus includes a coating for lubricating the insertion of the apparatus through the tracheal wall.

Typically at least one of the dilator and the sleeve is substantially rigid and curved.

Typically an outer diameter of the sleeve is suitable for receiving the tracheal tube of a standard size.

Typically the standard size includes an inner diameter that is any one of the following:
a) 6 mm;
b) 7 mm;
c) 8 mm;
d) 9 mm; and,
e) 10 mm.

In a second broad form the present invention seeks to provide a method for providing an airway through a tracheal wall of a subject using an apparatus including a dilator including a handle, wherein the dilator provides a channel extending from a proximal end to a distal end, and a sleeve detachably mounted to the dilator, the method including:
a) Holding the handle of the dilator to manipulate at least the dilator;
b) At least partially inserting the apparatus and a tracheal tube through the tracheal wall, wherein the dilator is for dilating a hole to allow insertion of the tracheal tube, and wherein the sleeve is for receiving the tracheal tube having an airway running from a proximal end to a distal end; and,
c) Detaching the tracheal tube from the sleeve to allow the dilator to be removed, thereby allowing the tracheal tube to provide an airway through the tracheal wall.

Typically the method includes inserting the apparatus over a guide extending through the tracheal wall, wherein the guide is provided into the channel and wherein the apparatus is guided by the guide.

Typically the guide includes a guide wire.

Typically the method includes engaging a detachment member thereby detaching the sleeve and the tracheal tube.

Typically the method includes positioning the detachment member between the proximal end of the tracheal tube and a flange on the sleeve, thereby urging apart the proximal end and the flange, and detaching the sleeve and the tracheal tube.

In a third broad form the present invention seeks to provide a kit for use in providing an airway through a tracheal wall of a subject, the kit including:
a) a dilator including a handle, wherein the handle allows a user to hold and manipulate at least the dilator, and wherein the dilator provides a channel extending from a proximal end to a distal end; and
b) at least two sleeves, wherein each sleeve is for detachably mounting to the dilator, each sleeve being for receiving a different size of a tracheal tube having an airway running from a proximal end to a distal end, and in use the dilator can be used to insert the tracheal tube through the tracheal wall with the tracheal tube being detachable from the sleeve to allow the dilator to be removed so that the tracheal tube provides an airway through the tracheal wall.

Typically the kit further includes at least one of the following:
a) A surgical drape;
b) A scalpel;
c) A needle;
d) A syringe;
e) A pre-dilator;
f) A guide wire;
g) An apparatus to dispose of a needle;
h) Lubricating gel; and,
i) A surgical swab.

Typically the kit further includes at least one tracheal tube.

It will be appreciated that different forms of the invention can be used interchangeable and/or in conjunction, depending on the implementation.

### BRIEF DESCRIPTION OF THE DRAWINGS

An example of the present invention will now be described with reference to the accompanying drawings, in which:—

FIGS. 1A and 1B are schematic diagrams of a first example of an apparatus for use in providing an airway through a tracheal wall of a subject, including a dilator and a sleeve;

FIGS. 2A and 2B are schematic diagrams of an example of a tracheal tube;

FIGS. 3A, 3B, 3C, 3D, 3E, 3F, 3G, 3H, and 3I are further schematic diagrams of the second example of an apparatus for use in providing an airway through a tracheal wall of a subject, including a sleeve and dilator including a handle;

FIGS. 4A, 4B, 4C, 4D, and 4E are schematic diagrams of a third example of an apparatus for use in providing an airway through a tracheal wall of a subject including a detachment member;

FIGS. 5A, 5B, and 5C are schematic diagrams of a fourth example of an apparatus for use in providing an airway through the tracheal wall of the subject; and,

FIG. 6 is a flow chart outlining an example method of use of the apparatus for use in providing an airway through the tracheal wall of the subject.

### DETAILED DESCRIPTION OF THE PREFERRED EMBODIMENTS

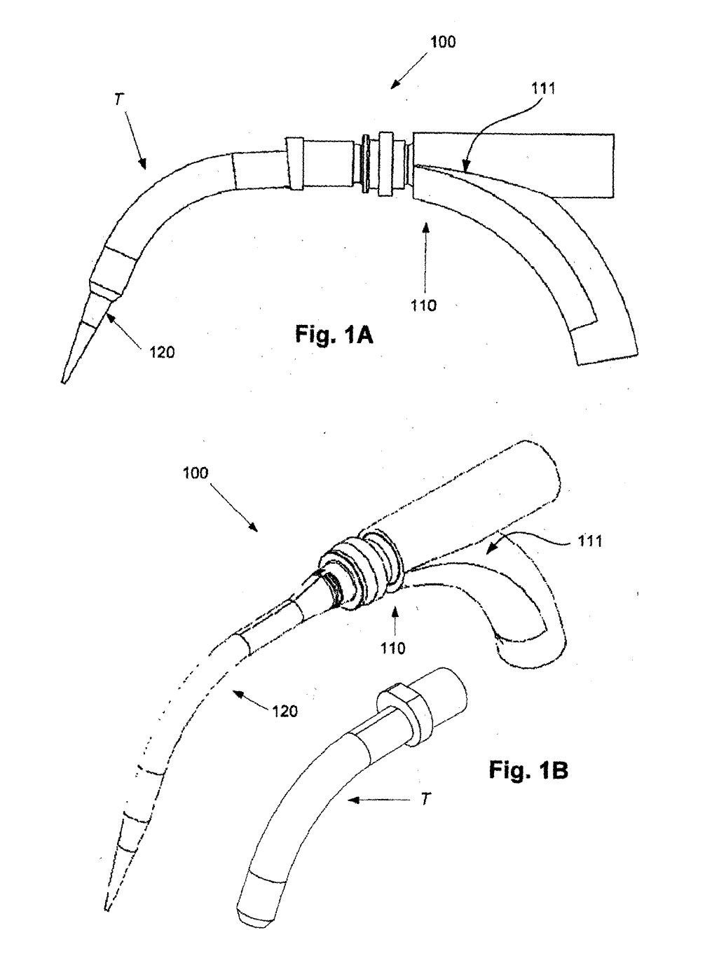

An example of an apparatus for use in providing an airway through a tracheal wall of a subject will now be described with reference to FIGS. 1A and 1B.

In this example, the apparatus 100 includes a dilator 110 including a handle 111, to allow a user to hold and manipulate at least the dilator 110.

The dilator 110 provides a channel extending from a proximal end to a distal end. The apparatus further includes a sleeve 120 that is detachably mounted to the dilator 110, the sleeve 120 being for receiving a tracheal tube T having an airway running from a proximal end to a distal end. An example tracheal tube T is shown provided on the apparatus 100 in FIG. 1A, and separate from the apparatus 100 in FIG. 1B.

In use the dilator 110 can be used to insert the tracheal tube T through the tracheal wall with the tracheal tube T being detachable from the sleeve 120 to allow at least the dilator 110 to be removed so that the tracheal tube T provides an airway through the tracheal wall.

Accordingly, the above described arrangement provides apparatus 100 for use in providing an airway through a tracheal wall of a subject, such as during a cricothyrotomy or tracheostomy procedures, or the like.

In this regard, the apparatus 100 may be provided in a kit, such as a surgical procedure kit or disposable kit, which can be used for performing a cricothyrotomy and/or tracheostomy procedure or the like. Typically, when performing such procedures it is for a range of subjects, including subjects of different sizes, such as small infants and large adults, and/or subjects of different genders, i.e. males and females. Practitioners then generally use a number of different dilators to progressively dilate a hole until it is sufficiently large to receive the required tracheal tube. In contrast, the current kit allows a user to perform either a cricothyrotomy procedure or tracheostomy procedure using a single dilator, with sleeves 120 of different sizes being provided to allow different sized tracheal tubes to be mounted on the dilator 110, which can then be used in a single step to both dilate a hole and insert the tube. However, a kit is not essential, and the apparatus may not be provided in a kit.

Thus, apparatus 100 can be used with standard tracheal tubes, and in this regard the apparatus 100 does not require a bespoke or adapted tracheal tube T. It will be appreciated that this has several advantages including allowing users to use standard/existing tracheal tubes, with which they are familiar, and have a history of use. Furthermore, existing and/or standard tracheal tubes have typically already acquired the necessary approvals of any regulatory bodies and/or satisfied any standard requirements. For example, the apparatus 100 could be used with standard and/or existing tracheal tubes that has a valid Food and Drug Administration (FDA) product code or insurance reimbursement code.

The apparatus 100 provides a number of further advantages including decreasing the number of dilators required during a procedure and thus reducing the number of insertions into, and removals from, the tracheal wall. It will be appreciated this can increase time efficiency in performing the procedure, as well as minimise the risks of contamination and infection associated with the introduction of a plurality of foreign objects into a subject.

The use of a handle 111 for holding and manipulating the apparatus 100 provides the user with more control over the apparatus 100, increasing the precision of use and reducing the risk of mishandling. It will further be appreciated that a user can easily control the insertion of the apparatus via the handle 111 with one hand, with the other hand available for operating detachment or retraction mechanisms and the like, however this is not essential and the user may control the insertion of the apparatus via the handle 111 with two hands.

A number of further features will now be described.

It will be appreciated that the sleeve 120 may not be provided as a contiguous member, and instead may include at least one of a separate mounting member and an elongated portion including a tapered portion, as will be discussed further below.

It will be appreciated that the sleeve 120 may be mounted to the dilator 110 in any appropriate manner, including an interference fit, friction fit, clip fit, or the like. It will further be appreciated that the tracheal tube T may be provided on the sleeve 120 in any suitable manner, including an interference fit, friction fit, clip fit, or the like.

In addition, a different size of sleeve 120 may be used to couple to a different size of tracheal tube T, such that a single dilator 110 can be used with different tracheal tubes of a range of sizes, including standard or non-standard sizes. It will be appreciated that this would allow a single dilator to be used for patients of varying sizes, with varying tracheal tube requirements. For example, a large male adult may require a tracheal tube with an inner diameter of 9 mm, whereas a small adult female may require a tracheal tube with an inner diameter of 6 mm. In this regard, an outer diameter of the sleeve would complement the inner diameter of the tracheal tube. Other standard sizes of the inner diameter of the tracheal tube include 7 mm, 8 mm, and 10 mm, however it will be appreciated that any suitable size of tracheal tube may be used.

Furthermore, it will be appreciated that at least the dilator 110 may be reused. In this respect, the dilator 110 and/or sleeve may be composed of a material suitable to undergo a sterilisation process, such as autoclaving, or any other sterilisation including through the use of heat, irradiation, chemicals, high pressure, or the like. However this feature is not essential and the dilator may instead be composed of any other material suitable for medical devices, including disposable medical devices, as will be discussed below.

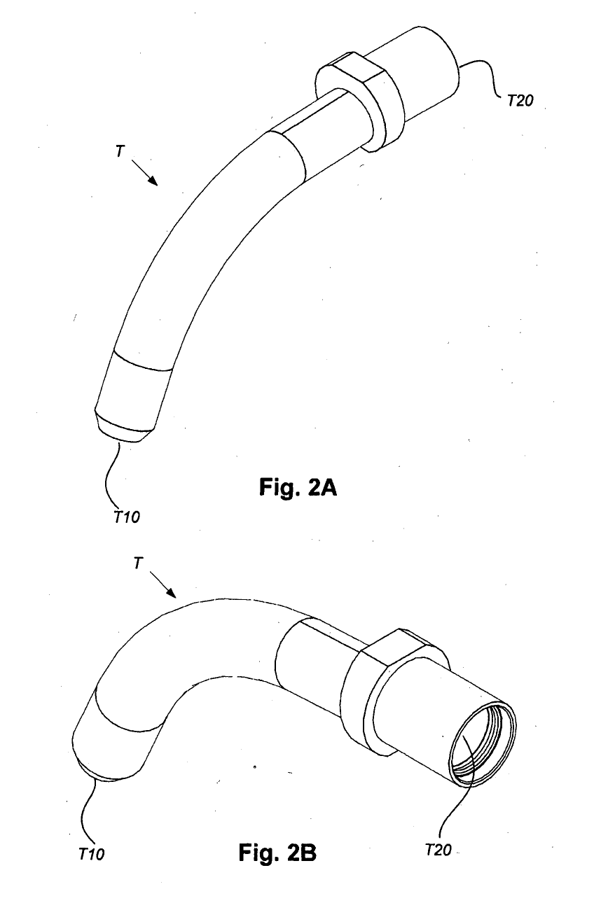

FIGS. 2A and 2B show a tracheal tube T having a proximal end T20 and a distal end T10. As the tracheal tube T is not part of the apparatus 100, it will be appreciated that the various features of the tracheal tube T are known and hence will not be described in any further detail.

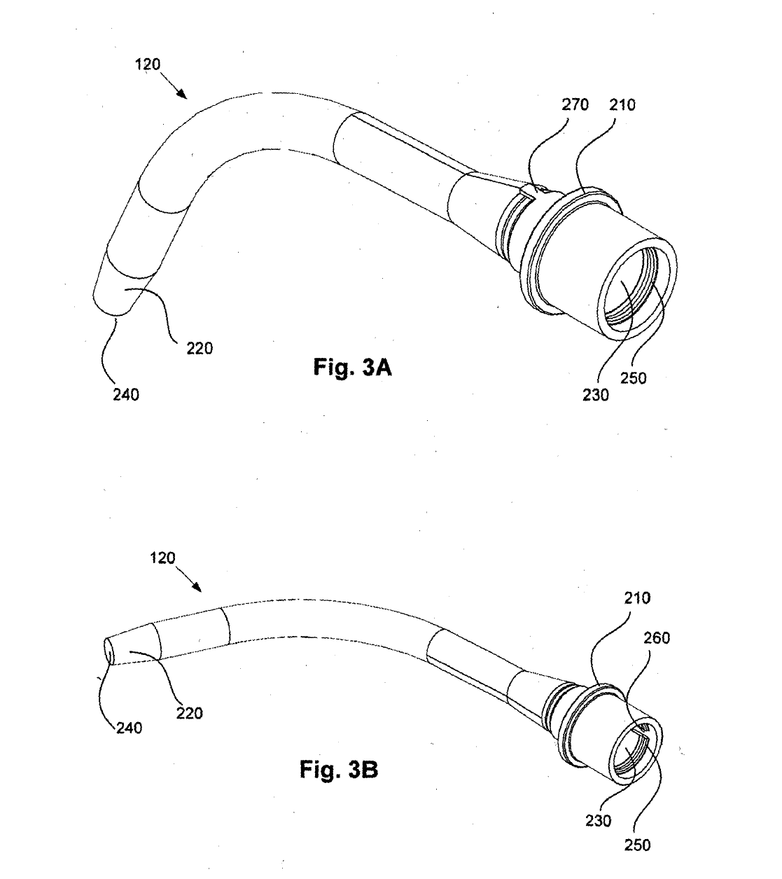

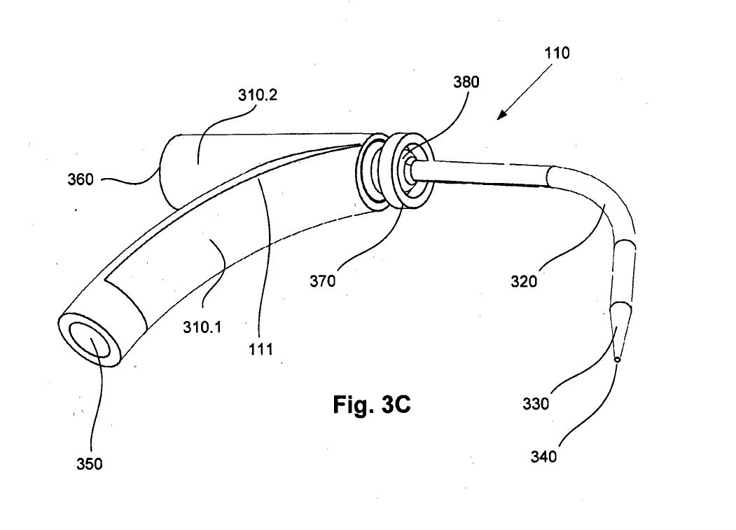

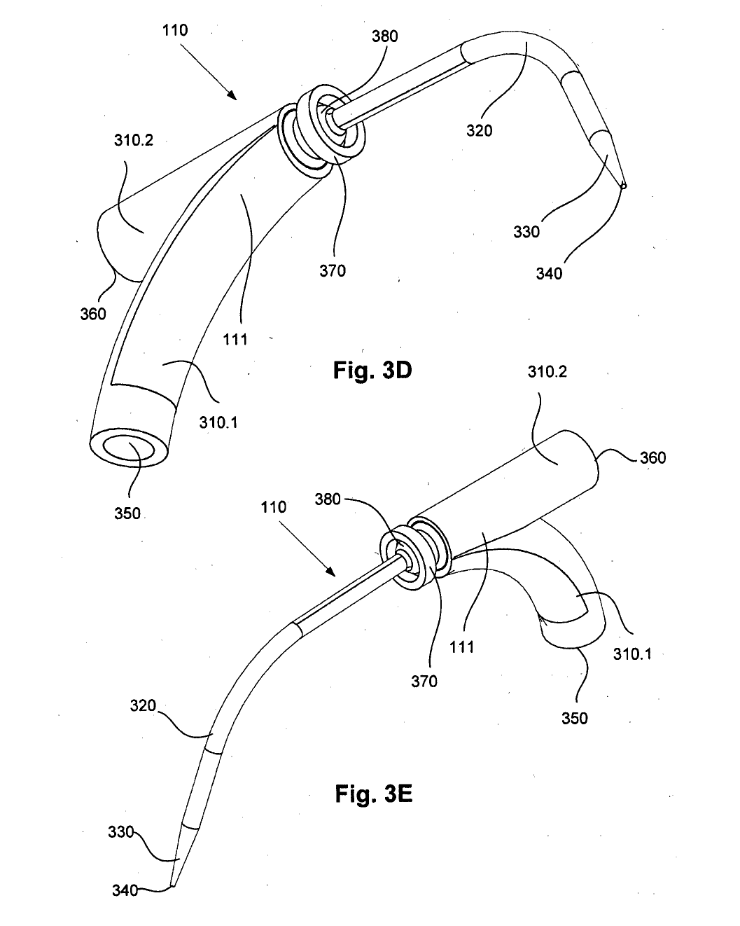

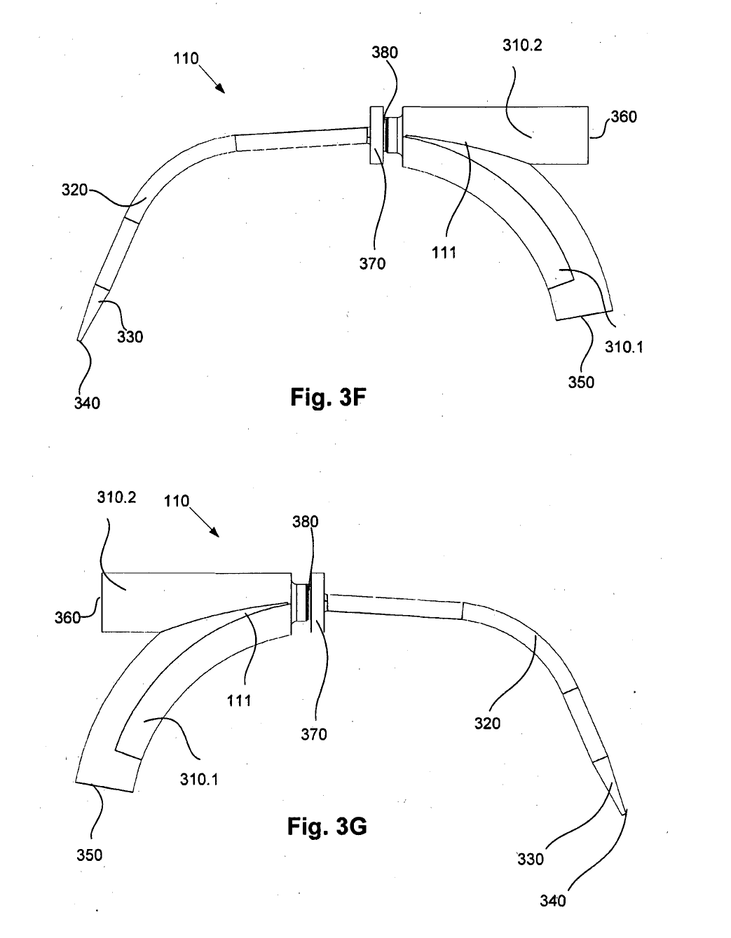

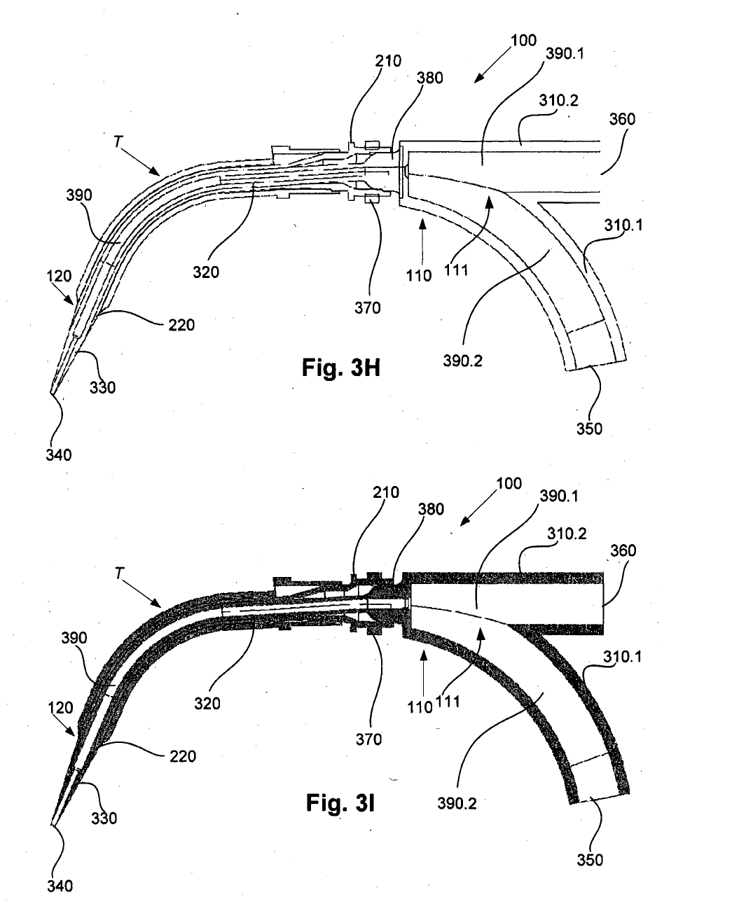

A second example apparatus for use in providing an airway through a tracheal wall of a subject is shown in FIGS. 3A to 3I. Features similar to those of the example apparatus described above have been assigned correspondingly similar reference numerals.

FIGS. 3A and 3B show a sleeve 120 including a proximal end 230 and a distal end 240, for receiving a tracheal tube T having an airway running from a proximal end to a distal end. In this regard, the sleeve 120 may receive the tracheal tube T in any suitable manner, including an interference fit, friction fit, clip fit, or the like.

The sleeve 120 may include a tapered portion 220 at the distal end, as will be described in more detail below. Furthermore, an inner surface of the proximal end 230 of the sleeve 120 may include an indented portion 260 and/or corrugations 250 or grooves or the like, as will be described in more detail below.

In this example, a flange 210 is provided on the sleeve 120 in order to ensure that the proximal end of the tracheal tube T is located below the flange 210 on the sleeve 120 when the tracheal tube T is mounted thereon. In this regard, the flange 210 enables the tracheal tube T to be provided at the correct position along the sleeve 120, such that detachment of the sleeve 120 from the tracheal tube T is not made overly difficult. Furthermore, the correct positioning of the tracheal tube T along the sleeve 120 may help ensure the apparatus 100 is not inserted too far into a subject's trachea. However, this feature is not essential.

Additionally or alternatively, the flange may provide a support during detachment of the sleeve 120 and tracheal tube T, or sleeve 120 and dilator 110, and this will be discussed further below.

Accordingly, the sleeve 120 can be formed from bio-compatible materials such as medical grades of metal, plastic or silicone, or a combination thereof. In this example, the sleeve 120 is a substantially rigid curved type, such that the shape of the sleeve 120 is maintained throughout the procedure. However, it will be appreciated that other types of sleeve 120, such as a substantially flexible type, can be used.

In this example, an alignment member 270 is provided on the sleeve 120, such that when a tracheal tube T is detachably provided on the sleeve 120, the alignment member 270 secures the tracheal tube T to the sleeve and/or prevents rotation of the tracheal tube T around the sleeve. However, this feature is not essential, and other mechanisms for securing the tracheal tube and/or preventing rotation could be used, for example corrugations on an outer surface of the sleeve 120, or the like.

In FIGS. 3C to 3G of this example, a dilator 110 is shown which includes a body 320 and a handle 111. In this example, the handle 111 includes a straight portion 310.2 and a curved portion 310.1 in order to ergonomically accommodate a user's hand while holding and manipulating the apparatus. In one example, one portion defines a grip which can be contoured to allow a user to comfortably and steadily hold and manipulate the apparatus. However, this feature is not essential, and the handle 111 could include a single straight or curved portion, a complementary finger grip profile, and the like.

It will be appreciated that the body 320 and handle 111 may be integrally formed together, permanently attached, or detachable. In this regard, the body 320 and handle 111 may be formed from the same, similar or different materials. The dilator 110 can be formed from any suitable bio-compatible materials such as medical grades of metal, plastic or silicone, or a combination thereof. In this example, the body 320 of the dilator 110 is a substantially rigid curved type, such that the shape of the body 320 is maintained throughout the procedure. However, it will be appreciated that other types of body 320, such as a substantially flexible type, can be used.

In this example, the body 320 of the dilator includes an elongated shaft including a dilating portion 330 for dilating the tracheal wall of the subject. For example, the dilating portion 330 can be provided as a tapered end of the body 320 with a substantially frustoconical shape, so that a diameter of the dilating portion 330 passing into the hole in the tracheal wall progressively increases as the dilator 110 is inserted, thus gradually dilating the hole during insertion. In one example, the dilating portion 330 extends from the distal end 340 of the dilator 110 by a length selected to allow the taper in the diameter of the dilating portion 330 to be sufficiently gradual so that trauma to the subject during insertion of the dilator 110 is substantially reduced.

Furthermore, when the sleeve 120 is provided over the dilator 110, the tapered portion 220 of the sleeve 120 is substantially adjacent to, or partially overlapping, the dilating portion 330 of the dilator 110, such that the dilating portion 330 aligns with the tapered portion 220. In this regard, during insertion of the apparatus 100 into the tracheal wall, the dilating portion 330 and tapered portion 220 continuously increase in a diameter of the hole in the tracheal wall to a sufficient size, and sufficiently gradually so that trauma to the subject during insertion of the apparatus 100 is substantially reduced.

An outer surface of the dilator 110 and/or sleeve 120 and/or tracheal tube T can be provided with a coating for lubricating the insertion of the tracheal tube T through the tracheal wall. It will be appreciated that the coating could be provided on the entire outer surface, or portions of the outer surface, such as the outer surface of the dilating portion 330. Such a coating can include a biologically compatible substance that, when wet, becomes slippery and therefore helps to allow the tracheal tube T to be easily passed through the tracheal wall. For example, the dilator 110 can have a hydrophilic coating, such as “PHOTO-LINK” coating material commercially available from SurModics, Inc., which can be activated by being dipped into sterile, saline or water. Once hydrated, the coating on the dilator 110 is made very slippery without the need for additional lubricants. Alternatively, the outer surface of the dilator 110 and/or sleeve 120 and/or tracheal tube T can be coated with a low friction material such as polytetrafluoroethylene (commonly known as “TEFLON™”) or the like. However, it will be appreciated that any suitable coating can be used.

In any event, a coating such as those described above will help to reduce the force required during the insertion of the dilator 110, the sleeve 120 and the tracheal tube T through the tracheal wall.

In this example, the dilator 110 includes a shoulder 380 that defines a mounting for receiving the sleeve 120. In this regard, the shoulder 380 may be formed of any suitable shape to receive the sleeve 120, such as a shape that complements the inner shape of a proximal end of the sleeve 120.

For example, the shoulder 380 may include a substantially annular shape and/or a graduated profiled and/or corrugated profile or the like. In this regard, it will be appreciated that a graduated profile allows the sleeve 120 to be mounted over the dilator 110 with a force sufficient to ensure the sleeve 120 does not detach from the dilator 110 until desired. A corrugated profile, or grooves along the shoulder 380, may be complemented by grooves or corrugations 250 inside the proximal end of the sleeve 120, which engage in order to secure the sleeve 120 over the dilator 110, until detachment is desired.

Furthermore, the shoulder 380 and sleeve 120 may include a complementary feature such as a lockable member with release button, a complementary cross-sectional portion, or the like, to prevent the sleeve 120 from rotating around the dilator 110 when detachably mounted thereon. For example, the shoulder 380 may include a raised portion (not shown) extending radially outwardly from the shoulder 380, and which complements an indented portion 260 on the sleeve 120, such that when sleeve 120 is mounted on the dilator 110, the raised portion (not shown) and indented portion 260 are aligned and the sleeve 120 is prevented from rotating around the dilator 110.

Additionally, the dilator 110 may include a collar 370 for use in detaching the sleeve 120 from the dilator 110, and this will be discussed further below.

Further to this example, FIGS. 3H and 3I provide a cross-sectional view of the apparatus 100 with tracheal tube T mounted thereon. In this example, a channel 390, 390.1 extends from a proximal end 360 of the straight portion 310.2 of the handle 111. However the channel 390, 390.2 could additionally or alternatively extend from a proximal end 350 of the curved portion 310.1 of the handle 111. The channel 390, 390.1 and/or 390, 390.2 is provided to allow aspiration throughout the insertion process as well as to receive a guide, configured to extend from the distal end 340 of the dilator 110 to the proximal end 350 or 360, through the channel 390, 390.1 and/or 390, 390.2. This is performed to allow insertion of the apparatus through the tracheal wall to be guided by a guide such as a guide wire, cannula, or the like, as will be described in more detail below. However, the use of a guide is not essential.

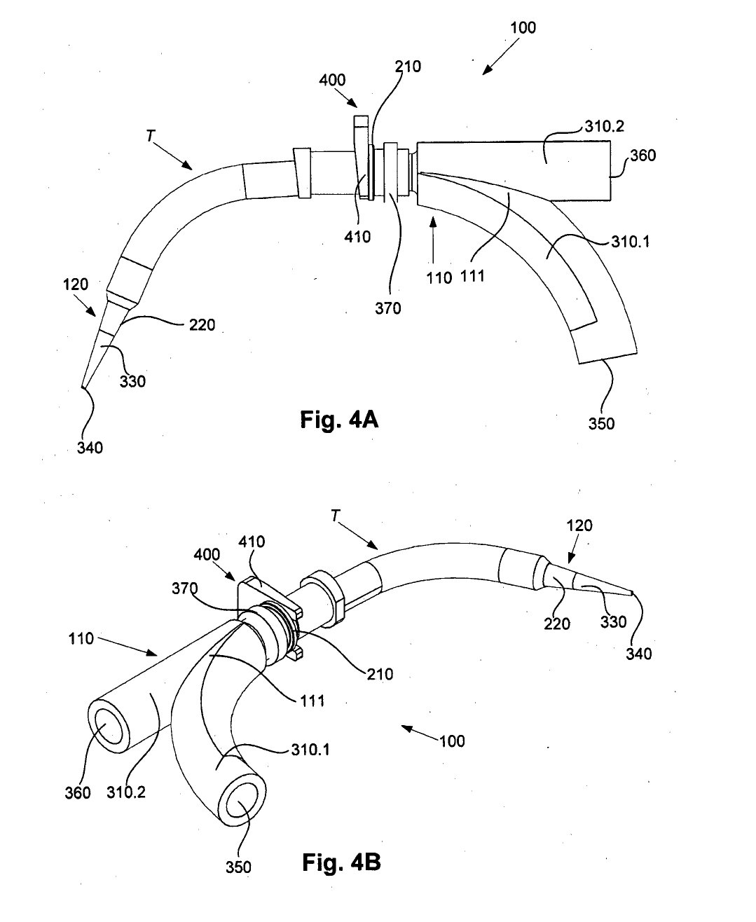

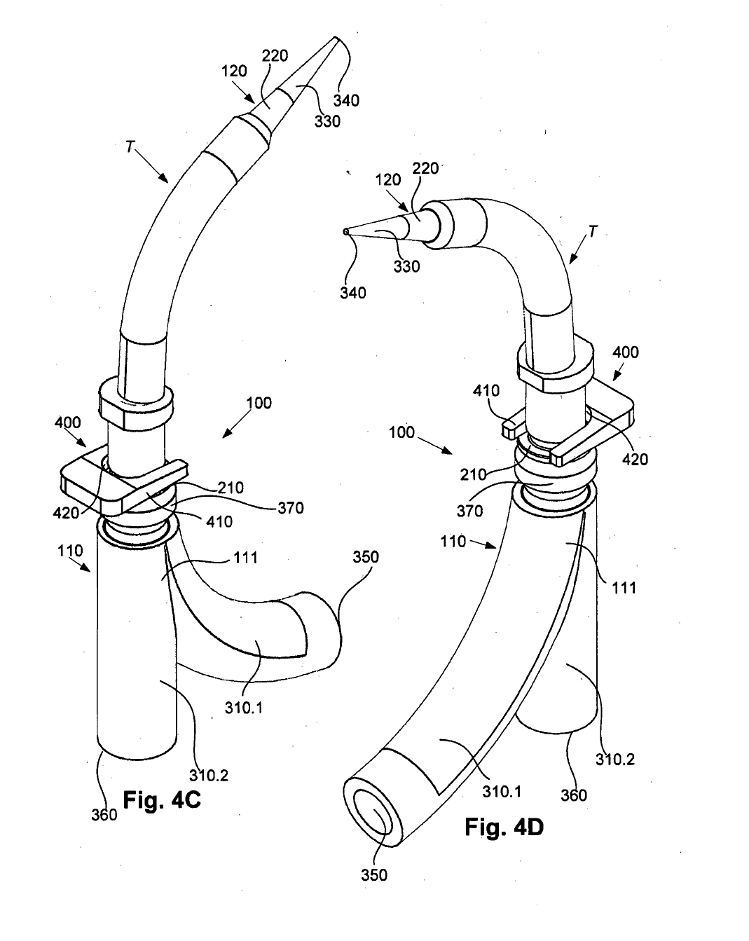

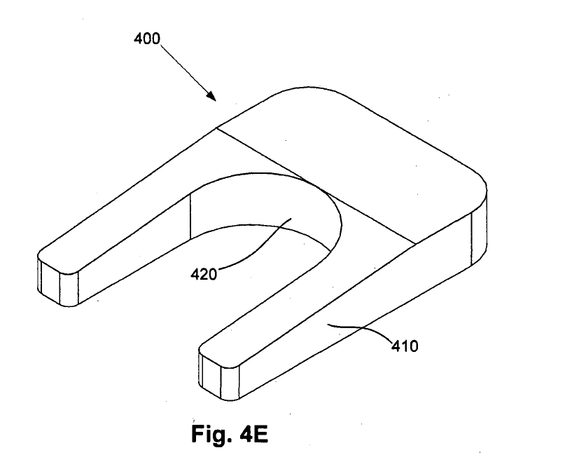

A third example apparatus for use in providing an airway through a tracheal wall of a subject will now be described with reference to FIGS. 4A to 4E. Features similar to those of the example apparatus described above have been assigned correspondingly similar reference numerals.

In this example, FIGS. 4A to 4D show an apparatus 100 for use in providing an air way through a tracheal wall of a subject, and a tracheal tube T. In this respect, the apparatus 100 includes a dilator 110, including a handle 111, to allow the user to hold and manipulate the dilator 110. The handle 111 further includes a straight portion 310.2 and curved portion 310.1, however as discussed above this feature is not necessary, and other arrangements of handle 111 could be used.

In this example, the dilator 110 includes a channel extending from proximal end 360 to a distal end 340, and a channel extending from a proximal end 350 to a distal end 340. The channels are provided to allow aspiration during the insertion procedure, and may additionally accommodate a guide wire, cannula, or the like. The dilator 110 further includes a body which in turn includes an elongated shaft and a dilating portion 330 for dilating the tracheal wall of the subject.

The apparatus 100 also includes a sleeve 120 detachably mounted on the dilator 110. Additionally, the sleeve 120 includes a tapered portion 220 at the distal end, for use in assisting dilation of the tracheal wall of the subject, as discussed above, and a flange 210 for assisting correct placement of the tracheal tube T and/or for enabling detachment of the sleeve 120 from the tracheal tube T.

In this example, a detachment member 400 is included that assists with detachment of the sleeve 120 and the tracheal tube T. For example, in order to detach the tracheal tube T and the sleeve 120, the detachment member 400 can be positioned between the tracheal tube T and the flange 210, as shown in FIGS. 4A to 4D, and pushed down by the user thereby urging apart the tracheal tube T and the flange 210 and detaching the sleeve 120 and the tracheal tube T.

In this regard, the detachment member 400 may be provided separately from the apparatus 100, or attached to the sleeve 120, or integrally formed with the sleeve 120.

It will be appreciated, that the detachment member 400 could be of any suitable shape. In the example of FIG. 4E, the detachment member 400 is substantially “U” shaped 420, such that when positioned between the tracheal tube T and the flange 210 the detachment member 400 substantially encloses a majority of the cross section of the apparatus. Furthermore, the detachment member 400 may be provided with a graduated or tapered cross section 410 so that in use the detachment member 400 can be gradually provided between the tracheal tube T and the flange 210, hence gradually urging apart the tracheal tube T and the flange 210, in order to increase usability and minimise the risk of breakage of the detachment member 400 or apparatus 100.

However, it will be appreciated that any suitable arrangement of detachment member 400 may be used, for example, a hook shaped arrangement for positioning under the proximal end of the tracheal tube T to detach from the sleeve 120. The detachment member 400 is also not an essential feature, and other detachment operations may be used for detaching the sleeve 120 and tracheal tube T, as previously discussed.

Additionally or alternatively, the detachment member 400 may also be used in detaching the collar 120 from the dilator 110. In this regard, the detachment member 400 is positioned between the flange 210 and the collar 370 of the dilator 110 in order to urge the flange 210 and the collar 370 apart thereby detaching the collar 120 from the dilator 110. However, this feature is not essential and it will be appreciated that other mechanisms for detaching the sleeve 120 from the dilator 110 may be used, as discussed above.

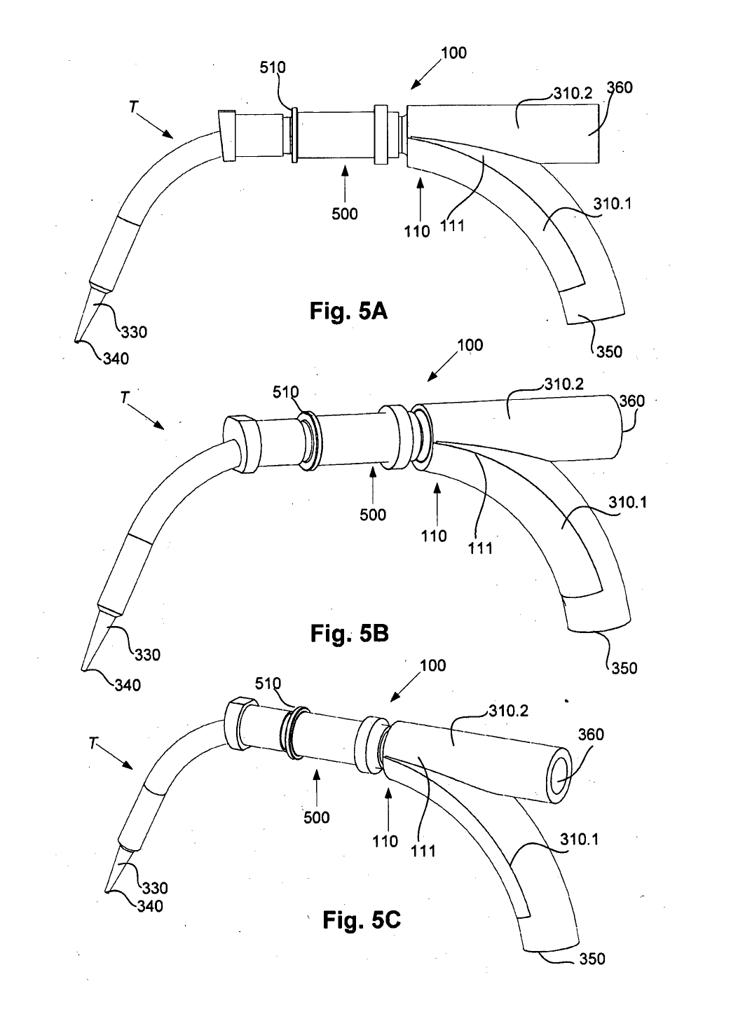

A further example of an apparatus 100 for use in providing an airway through the tracheal wall of a subject is provided with reference to FIGS. 5A, 5B and 5C. Features similar to those of the apparatus described above are identified with similar reference numerals.

The apparatus 100 includes a dilator 110 and a different size of tracheal tube T from previous examples mounted on the apparatus 100.

In this example, the dilator 110 includes a handle 111, to allow the user to hold and manipulate the at least the dilator 110. The handle 111 further includes a straight portion 310.2 and curved portion 310.1, however as discussed above this feature is not necessary, and other arrangements of handle 111 could be used.

In this example, the dilator 110 includes a channel extending from proximal end 360 to a distal end 340, and a channel extending from a proximal end 350 to a distal end 340. The channels are provided to allow aspiration during the insertion procedure, and may additionally accommodate a guide wire, cannula, or the like. The dilator 110 further includes a body which in turn includes an elongated shaft and a dilating portion 330 for dilating the tracheal wall of the subject.

The apparatus 100 also includes a mounting member 500 mounted on the dilator 110 using any suitable method, such as described above with reference to mounting a sleeve 120 to the dilator 110. Additionally, the mounting member 500 includes a flange 510 for assisting correct placement of the tracheal tube T and/or for enabling detachment of the mounting member 500 from the tracheal tube T, also as discussed above with reference to mounting the tracheal tube T to the sleeve 120.

It will be appreciated, that in this example the mounting member 500 is used instead of a sleeve, as the tracheal tube T includes an inner diameter which is sufficiently small that it corresponds to an outer diameter of the dilator 110, and therefore may be mounted directly on the dilator 110 without requiring a sleeve 120. Furthermore, the mounting member 500 does not include a tapered portion, as the dilating portion 330 of the dilator 110 is sufficient to dilate the tracheal wall of the subject to a diameter of sufficient size for insertion of the tracheal tube T, without incurring unnecessary trauma to the tracheal wall.

It will further be appreciated, that the mounting member 500 may be detached from the dilator 110 by any suitable method, such that a sleeve 120 may be detachably mounted to the dilator 110, the sleeve 120 being for receiving a tracheal tube T having a larger inner diameter than the inner diameter of the tracheal tube T for mounting on the mounting member 500. In this regard, the mounting member 500 may be detached from the dilator 110, for example using any of the methods described above with reference to detaching the sleeve 120 from the dilator 110. The tracheal tube T may also be detached from the mounting member 500, for example using any of the methods described above with reference to detaching the tracheal tube T from the sleeve 120.

It will also be appreciated that a tapered portion, or elongated portion including a tapered portion, may be detachably connected to the mounting member 500, such that the arrangement of the tapered portion and mounting member 500 provides a sleeve.

An example of a method for providing an airway through the tracheal wall of a subject will now be described. The method includes using an apparatus 100 that includes a dilator 110 including a handle 111, in which the dilator 110 provides a channel extending from a proximal end to a distal end. The apparatus 100 further includes a sleeve 120 that is detachably mounted to the dilator 110.

The method includes holding the handle 111 of the dilator 110 to manipulate at least the dilator 110. The method further includes inserting at least part of the apparatus 100 and a tracheal tube T through the tracheal wall, such that the dilator 110 is for dilating a hole to allow insertion of the tracheal tube T, and the sleeve 120 is for receiving the tracheal tube T which has a airway running from a proximal end to a distal end. The method also includes detaching the tracheal tube T from the sleeve 120 to allow the dilator 110 to be removed, to allow the tracheal tube T to provide an airway through the tracheal wall.

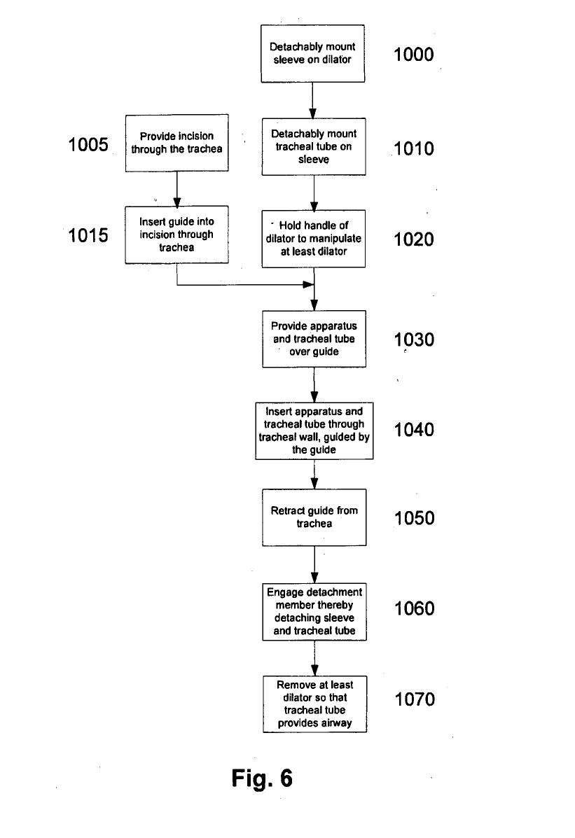

A further example of a method for providing an airway through a tracheal wall of the subject will now be described with reference to FIG. 6.

As apparatus 100 is for insertion into a part of the subject's body, it will be appreciated that standard preparation of the subject will necessary prior to the use of this apparatus. The following example method therefore presumes that the subject is adequately prepared as per preparations required when using other air way access apparatus.

In step 1005, an incision is provided through the trachea. Typically, such an incision may be created by making a horizontal or vertical incision using a scalpel in the skin at the chosen insertion site.

In step 1015, a guide, for example a guide wire, is advanced into the tracheal wall, using any suitable method. In one example, a needle and cannula with a syringe attached are inserted in to the midline of the insertion site, following the creation of the incision. The needle is advanced into the tracheal wall until entry of the needle and cannula into to the trachea is confirmed, by confirming aspiration, for example by detecting air bubbles in the syringe, or the like. The cannula may be left in place while the needle and syringe are retracted. A guide, for example a guide wire, is advanced through the cannula and into the tracheal wall before the removal of the cannula from the trachea.

In a further example, a pre-dilator may optionally be advanced into the insertion site, and subsequently removed, thus partially dilates the tracheal wall in preparation for insertion of the tracheal tube. It will be appreciated that the pre-dilator may optionally be guided by the guide wire. However, this step is not essential.

In another example, a bundle of a plurality of optical fibres can be used, and a proximal end of the bundle of optical fibres can be fitted with a viewing lens to allow an image to be observed by the user that is indicative of the position of the distal end of the bundle of optical fibres. It will be appreciated that this is similar to the functionality of an endoscope, and accordingly, similar imaging techniques can be used, such as the use of an optical detector, or camera, allowing the inside of the trachea to be imaged. This allows the operator to view the positioning of the needle, cannula, apparatus 100, and/or tracheal tube T, for example on a video display, thereby allowing the user to confirm the correct positioning of the needle, cannula, apparatus 100, and/or tracheal tube T, or the like, in the tracheal wall.

At step 1000, a sleeve 120 is detachably mounted to a dilator 100, using any suitable method and arrangement, for example as discussed above with reference to any of the preceding examples.

At step 1010 the tracheal tube T is detachably mounted on the apparatus 100, using any suitable method and arrangement, for example as discussed above with reference to any of the preceding examples.

At step 1020 the user holds the handle 111 of the dilator 110 to control the apparatus 100 and tracheal tube T. Accordingly, holding and manipulating the apparatus 100 using the handle 111 provides the user with more control over the apparatus 100, increasing the precision of use and reducing the risk of mishandling. It will further be appreciated that a user can easily control the insertion of the apparatus via the handle 111 with one hand, with the other hand available for operating detachment or retraction mechanisms and the like.

It will be appreciated that steps 1000, 1010, and optionally 1020, and steps 1005 and 1015 can be performed in parallel, or sequentially in either order, however typically steps 1000, 1010, and 1020 are performed by a different practitioner and at the same time as steps 1005 and 1015. For example, a surgeon may perform steps 1005 and 1015, the incision and insertion of the guide, and a nurse may perform the steps 1000, 1010, and 1020 of assembling the apparatus 100 and tracheal tube T. However this is not essential, and steps 1000 and 1010 may occur at any suitable time, including immediately prior to the procedure, or during earlier preparations such as during the preparation of an operating theatre, or restocking of a paramedic kit. Alternatively, a kit may be provided, as discussed below, including a tracheal tube T pre-mounted to the apparatus 100 or sleeve 120 or dilator 110.

At step 1030, the apparatus 100 is provided over the guide, such that the distal end 340 of the dilator 110 is provided over the guide and the guide is fed through a channel 390, 390.1 and/or 390, 390.2 of the dilator 110.

At step 1040, the tracheal tube T and apparatus 100 are inserted through the tracheal wall with insertion of the tracheal tube T being controlled by the user by gripping and moving the handles 111 in the appropriate direction. In one example, an outer surface of the tracheal tube T, dilator 110 and/or sleeve 120 include a coating for lubricating insertion, as discussed above. It will be appreciated that the dilating portion 330 of the dilator 110 is for dilating the hole through the tracheal wall as the apparatus 100 and tracheal tube T are inserted, for example, as described above.

It will be appreciated that the guide therefore operates to prevent the incision (or stoma or hole) in the tracheal wall, which was formed at step 1005, from closing before insertion of the apparatus 100. Furthermore, guiding the insertion of the apparatus 100 with the guide helps to simplify the insertion process, by aligning the apparatus 100 with the incision previously created, and thereby helps to reduce trauma to the subject compared to directly inserting the apparatus 100 through a hole in the tracheal wall without using a guide.

Once the tracheal tube T is adequately position through the tracheal wall, the guide is retracted from the trachea through the dilator 110, at step 1050. This prevents the guide from obstructing the airway. However, this is not essential.

At step 1060, the tracheal tube T is detached from the sleeve 120 using a detachment member 400. The detachment member 400 operates according to any suitable arrangement, for example, as previously disclosed with reference to the above examples.

At step 1070, the dilator 110 is removed from the tracheal wall, such that the tracheal tube T provides an airway through the tracheal wall. In a further example, the sleeve 120 is additionally removed from the tracheal wall. In this regard, it will be appreciated that the sleeve 120 may be mounted to the dilator 110 during removal, such that the apparatus 100 is removed intact, or alternatively the sleeve 120 may be detached from the dilator 110 and removed after the dilator 110 has been removed.

An unobstructed airway is now provided through the tracheal wall via the tracheal tube T, allowing the subject to breathe freely therethrough.

It will be appreciated that the subsequent use of the tracheal tube T may be in accordance with known practices. For example, a cuff balloon may be included with the tracheal tube, T, in order to ensure an adequate seal is provided in the subject's trachea, which assists the subject to comfortably breathe through the tracheal tube T. A tracheal tube flange and/or neck strap may be provided for securing the tracheal tube T at the desired position on the subject's trachea. Additionally, the tracheal tube T may be optionally connected to a ventilation system to provide a direct air or oxygen source to the subject. In this example, the proximal end T20 of the tracheal tube T is a standard connector which is compatible with standard ventilation system tubing, such that the ventilation system can be easily connected to the tracheal tube T.

Some variations in the order of the steps outlined above are possible without changing the overall operation of the apparatus. For example, the retraction of the guide may occur at the same time or after the detachment and removal of the dilator 110, and the sleeve 120 may be removed at the same time or subsequent to the removal of the dilator 110 from the trachea of the subject.

In a further example, a kit is provided for use in providing an airway through a tracheal wall of the subject. The kit includes a dilator 110 including a handle 111 wherein the handle 111 allows the user to hold a manipulator that leads the dilator 110 and in which the dilator 110 provides a channel extending from a proximal end 340 to a distal end 360. Also provided in the kit, two or more sleeves 120 in which each sleeve is for detachably mounting to the dilator 110. Each sleeve 120 is of a different size, and for receiving a different size of tracheal tube T.

The kit could additionally include a surgical drape, a scalpel, a needle, a syringe, a pre dilator, a guide wire, any apparatus suitable for the disposal of the needle, lubricating gel, and a surgical swab, or the like. Furthermore, the kit may include one or more tracheal tubes T, however this is not essential, and tracheal tubes T may be packaged and shipped separately.

It will be appreciated, that the kit may include the apparatus 100 and tracheal tube T in various states of assembly. For example, the kit may include one of the two or more sleeves 120 pre-mounted to the dilator 110. Furthermore, the kit may include a tracheal tube T pre-mounted to the apparatus 100, or dilator 110.

In this regard, if the pre-assembled components of the kit include a sleeve 120 or tracheal tube T of the correct size for the subject, a user has fewer steps to perform in providing an airway through the tracheal wall of the subject. In the event the pre-assembled components of the kit include a sleeve 120 or tracheal tube T that is not the correct size for the subject, a user detaches the tracheal tube T and/or sleeve 120 from the dilator and detachably mounts the sleeve 120 and/or tracheal tube T of the correct size for the subject.

Furthermore, the dilator 110 may be provided in the kit such that the smallest size of tracheal tube T for use with that dilator 110 may be mounted directly on the dilator 110. It will be appreciated that in this example, the kit may include the tracheal tube T pre-mounted to the dilator 110, or alternatively the tracheal tube T may be provided un-mounted to the dilator 110, or provided separate from the kit. In this regard, larger tracheal tubes T would require a sleeve 120 of a corresponding size, such that the sleeve 120 is detachably mounted to the dilator 110, and the tracheal tube is detachably mounted to the sleeve 120.

It will be appreciated that an apparatus with the above described examples, facilitates a simplified method for use in providing an airway through the tracheal wall of a subject. For example, the apparatus may be provided in a single kit, such a surgical procedure kit or disposable kit, providing the necessary equipment to perform a procedure on a range of subject and reducing the equipment typically required for such procedures. In turn, the reduction in equipment can decrease the time taken to perform the procedure, and well as minimise contamination and infection risks associated with the invasive insertion of a plurality of foreign objects into a subject. The apparatus can be used with standard tracheal tubes, thus allowing practitioners to use familiar tracheal tubes, and negating the need for additional approvals of the tracheal tubes, for example from regulatory bodies. Furthermore, providing a handle allows a user more control thus minimising the risk of mishandling of the apparatus, and allowing for greater precision during use. Additionally, the apparatus reduces

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers.

Persons skilled in the art will appreciate that numerous variations and modifications will become apparent. All such variations and modifications which become apparent to persons skilled in the art, should be considered to fall within the spirit and scope that the invention broadly appearing before described. Thus, for example, it will be appreciated that features from different examples above may be used interchangeably where appropriate.

1) An apparatus for use in providing an airway through a tracheal wall of a subject, the apparatus including:
a) a dilator including a handle, wherein the handle allows a user to hold and manipulate at least the dilator, and wherein the dilator provides a channel extending from a proximal end to a distal end; and
b) a sleeve detachably mounted to the dilator, the sleeve being for receiving a tracheal tube having an airway running from a proximal end to a distal end, and in use the dilator can be used to insert the tracheal tube through the tracheal wall with the tracheal tube being detachable from the sleeve to allow the dilator to be removed so that the tracheal tube provides an airway through the tracheal wall.

2) An apparatus according to claim 1, wherein the dilator includes a shoulder, wherein the shoulder defines a mounting for receiving the sleeve.

3) An apparatus according to claim 2, wherein the shoulder includes at least one of the following:
a) A substantially annular shape;
b) A graduated profile; and,
c) A substantially corrugated profile.

4) An apparatus according to claim 1, wherein the dilator further includes a body including a dilating portion for dilating the tracheal wall of the subject.

5) An apparatus according to claim 4, wherein the body includes an elongated shaft.

6) An apparatus according to claim 4, wherein the dilating portion is substantially frustoconical.

7) An apparatus according to claim 1, wherein the sleeve is mounted to the dilator using any one of:
a) an interference fit;
b) a friction fit; and,
c) a clip fit.

8) An apparatus according to claim 1, wherein the sleeve includes a tapered portion for dilating the tracheal wall of the subject.

9) An apparatus according to claim 8, wherein the tapered portion aligns with a dilating portion of the dilator, wherein the tapered portion and the dilating portion are for dilating the tracheal wall of the subject.

10) An apparatus according to claim 1, wherein the apparatus includes a detachment member, wherein the detachment member enables detachment of the sleeve and the tracheal tube.

11) An apparatus according to claim 10, wherein the sleeve includes a flange, wherein in use the detachment member is positioned between the tracheal tube and the flange, thereby urging apart the tracheal tube and the flange and detaching the sleeve and the tracheal tube.

12) An apparatus according to claim 10, wherein the detachment member includes a substantially graduated profile.

13) An apparatus according to claim 10, wherein the detachment member is substantially “U” shaped.

14) An apparatus according to claim 10, wherein the detachment member enables detachment of the sleeve and the dilator.

15) An apparatus according to claim 14, wherein the dilator includes a collar and wherein, in use, the detachment member is positioned between the sleeve and the collar, thereby urging apart the sleeve and the collar and detaching the sleeve and the dilator.

16) An apparatus according to claim 10, wherein the detachment member is any one of the following:
a) detached from the apparatus;
b) attached to any one of the sleeve and the dilator; and,
c) integrally formed with any one of the sleeve and the dilator.

17) An apparatus according to claim 1, wherein the apparatus includes a guide provided through the channel, thus allowing insertion of the apparatus through the tracheal wall to be guided by the guide.

18) An apparatus according to claim 17, wherein the guide includes a guide wire.

19) An apparatus according to claim 1, wherein an outer surface of the apparatus includes a coating for lubricating the insertion of the apparatus through the tracheal wall.

20) An apparatus according to claim 1, wherein at least one of the dilator and the sleeve is substantially rigid and curved.

21) An apparatus according to claim 1, wherein an outer diameter of the sleeve is suitable for receiving the tracheal tube of a standard size.

22) An apparatus according to claim 21, wherein the standard size includes an inner diameter that is any one of the following:
a) 6 mm;
b) 7 mm;
c) 8 mm;
d) 9 mm; and,
e) 10 mm.

23) A method for providing an airway through a tracheal wall of a subject using an apparatus including a dilator including a handle, wherein the dilator provides a channel extending from a proximal end to a distal end, and a sleeve detachably mounted to the dilator, the method including:
a) Holding the handle of the dilator to manipulate at least the dilator;
b) At least partially inserting the apparatus and a tracheal tube through the tracheal wall, wherein the dilator is for dilating a hole to allow insertion of the tracheal tube, and wherein the sleeve is for receiving the tracheal tube having an airway running from a proximal end to a distal end; and,
c) Detaching the tracheal tube from the sleeve to allow the dilator to be removed, thereby allowing the tracheal tube to provide an airway through the tracheal wall.

24) A method according to claim 23, wherein the method includes inserting the apparatus over a guide extending through the tracheal wall, wherein the guide is provided into the channel and wherein the apparatus is guided by the guide.

25) A method according to claim 24, wherein the guide includes a guide wire.

26) A method according to claim 23, wherein the method includes engaging a detachment member thereby detaching the sleeve and the tracheal tube.

27) A method according to claim 26, wherein the method includes positioning the detachment member between the proximal end of the tracheal tube and a flange on the sleeve, thereby urging apart the proximal end and the flange, and detaching the sleeve and the tracheal tube.

28) A kit for use in providing an airway through a tracheal wall of a subject, the kit including:
a) a dilator including a handle, wherein the handle allows a user to hold and manipulate at least the dilator, and wherein the dilator provides a channel extending from a proximal end to a distal end; and
b) at least two sleeves, wherein each sleeve is for detachably mounting to the dilator, each sleeve being for receiving a different size of a tracheal tube having an airway running from a proximal end to a distal end, and in use the dilator can be used to insert the tracheal tube through the tracheal wall with the tracheal tube being detachable from the sleeve to allow the dilator to be removed so that the tracheal tube provides an airway through the tracheal wall.

29) A kit according to claim 28, wherein the kit further includes at least one of the following:
a) A surgical drape;
b) A scalpel;
c) A needle;
d) A syringe;
e) A pre-dilator;
f) A guide wire;
g) An apparatus to dispose of a needle;
h) Lubricating gel; and,
i) A surgical swab.

30) A kit according to claim 28, wherein the kit further includes at least one tracheal tube.

31) (canceled)

32) (canceled)
